(19) United States
(12) Patent Application Publication
(10) Pub. No.: US 2005/0273872 A1
(43) Pub. Date: Dec. 8, 2005

(54) Protein production in transgenic avians

(75) Inventors: Helen Sang, Midlothian (GB); Michael McGrew, Midlothian (GB); Adrain Sherman, Midlothian (GB); Karen Elizabeth Jervis, Midlothian (GB); William Howard Stimson, Midlothian (GB); Kyriacos Mitrophanous, Oxford (GB); Fiona Ellard, Oxford (GB); Alan Kingsman, Oxford (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignees: Viragen Incorporated, Plantation, FL (US); Oxford Biomedica (UK) LTD., Oxford (GB)

(21) Appl. No.: 10/536,550

(22) PCT Filed: Nov. 27, 2003
(86) PCT No.: PCT/GB03/05191

§ 371 Date: Jul. 25, 2005

(30) Foreign Application Priority Data
Nov. 27, 2002 (GB) 0227645.9

(51) Int. Cl. A01K 67/027

(52) U.S. Cl. 800/19; 800/21; 119/300

(57) ABSTRACT

The present invention relates to the generation of transgenic avians and the production of recombinant proteins. More particularly, the invention relates to the enhanced transduction of avian cells by exogenous genetic material so that the genetic material is incorporated into an avian genome in such a way that the modification becomes integrated into the germline and results in expression of the encoded protein within the avian egg.

The present invention relates to the generation of transgenic avians and the production of recombinant proteins. More particularly, the invention relates to the enhanced transduction of avian cells by exogenous genetic material so that the genetic material is incorporated into an avian genome in such a way that the modification becomes integrated into the germline and results in expression of the encoded protein within the avian egg.

The ability to manufacture large amounts of pharmaceutical grade proteins is becoming increasingly important in the biotechnology and pharmaceutical arenas. Recent successes of such products in the marketplace, especially those of monoclonal antibodies, have put an enormous strain on already stretched global manufacturing facilities. This heightened demand for manufacturing capacity, the consequential high premiums on capacity and the long wait for production space, plus the cost of and issues involved in producing proteins in cell lines, has prompted companies to look beyond traditional modes of production (Andersson & Myhanan, 2001). Traditional methods for manufacture of recombinant proteins include production in bacterial or mammalian cells. One of the alternative manufacturing strategies is the use of transgenic animals and plants for production of proteins.

It was by genetic engineering that the first genetically modified (transgenic) animal was produced, by transferring the gene for the protein of interest into the target animal. Current transgenic technology can be traced back to a series of pivotal experiments conducted between 1968 and 1981 including: the generation of chimeric mice by blastocyst injection of embryonic stem cells (Gardner, 1968), the delivery of foreign DNA to rabbit oocytes by spermatozoa (Brackett et al, 1971), the production of transgenic mice made by injecting viral DNA into pre-implantation blastocysts (Jaehisch & Mintz, 1974) and germline transmission of transgenes in mouse by pronuclear injection (Gordon & Ruddle, 1981). For the early part of transgenics' history, the focus was upon improving the genetic makeup of the animal and thus the yield of wool, meat or eggs (Curtis & Barnes, 1989; Etches & Gibbins, 1993,). However in recent years there has been interest in utilising transgenic systems for medical applications such as organ transplantation, models for human disease or for the production of proteins destined for human use.

A number of protein based biopharmaceuticals have been produced in the milk of transgenic mice, rabbits, pigs, sheep, goats and cows at reasonable levels, but such systems tend to have long generation times—some of the larger mammals can take years to develop from the founder transgenic to a stage at which they can produce milk. Additional difficulties relate to the biochemical complexity of milk and the evolutionary conservation between humans and mammals, which can result in adverse reactions to the pharmaceutical in the mammals which are producing it (Harvey et al, 2002).

There is increasing interest in the use of chicken eggs as a potential manufacturing vehicle for pharmaceutically important proteins, especially recombinant human antibodies. Huge amounts of therapeutic antibodies are required by the medical community each year, amounts which can be kilogram or metric tons per year, so a manufacturing methodology which could address this shortage would be a great advantage. Once optimised, a manufacturing method based on chicken eggs has several advantages as compared to mammalian cell culture or use of transgenic mammalian systems. Firstly, chickens have a short generation time (24 weeks), which would allow transgenic flocks to be established rapidly. The following table shows a comparison between the different types of transgenic systems. Secondly, the capital outlays for a transgenic animal production facility are far lower than that for cell culture. Extra processing equipment is minimal in comparison to that required for cell culture (BioPharm, 2001). As a consequence of these lower capital outlays, the production cost per unit of therapeutic will be lower than that produced by cell culture. In addition, transgenic systems provide significantly greater flexibility regarding purification batch size and frequency and this flexibility may lead to further reduction of capital and operating costs in purification through batch size optimisation. The third advantage of increased speed to market should become apparent when the technology has been developed to a commercially viable degree. Transgenic mammals are capable of producing several grams of protein product per litre of milk, making large-scale production commercially viable (Weck, 1999). Mammals do not have a significant advantage in terms of the time take to scale up production, since gestation periods for cows and goats are 9 months and 5 months respectively (Dove, 2000) and it can take up to five years to produce a commercially viable herd. However, once the herd is established, the yield of product from milk will be high.

| | | | | | Protein | Founder |
|---|---|---|---|---|---|---|
| | | Maturity/ | | Time to | (per litre/ | animal |
| | | Generation | Offspring | Production | egg per | development |
| Animal | Gestation | time | Produced | Herd/Flock | day) | cost |
| Cow | 9 months | 2 years | 1 per year | 5+ years | 15 g | $5-10 M |
| Goat | 5 months | 8 months | 2-4 per year | 3-5 years | 8 g | $3 M |
| Sheep | 5 months | 8 months | 2 per year | 3-5 years | 8 g | $2 M |
| Pigs | 4 months | 8 months | 10 | ? | 4.1 g | ? |
| Rabbits | 1 month | 5 months | 8 | ? | 0.05 g | ? |
| Chicken | 21 days | 6 months | 21 per month | 18 months | 0.3 g | $0.25 M |

A comparison between the various transgenic animal production systems (Dove, 2000).

The short generation time for birds also allows for rapid scale-up. The incubation period of a chicken is only 21 days and it reaches maturity within six months of hatch. Indeed, once the founder animals of the flock have been established, a flock can be established within 18 months (Dove, 2000). The process of scaling up the production capability should be simpler and far faster than a herd of sheep, goats or cows.

A further advantage rests in the fact that eggs are naturally sterile vessels. One of the inherent problems with cell culture methods of production is the risk of microbial contamination, since the nutrient rich media used tends to encourage microbial growth. Transgenic production offers a lower risk alternative, since the production of the protein will occur within the animal itself, whose own body will combat most infections. Chicken eggs provide an even lower risk alternative: the eggs are sealed within the shell and membrane and thus largely separated from the environment. The evolutionary distance between humans and birds means that few diseases are common to both.

Still a further potential advantage lies in the post-translational modification of chicken proteins. The issue of how well a production process can reproduce the natural sugar profile on the proteins which are produced, is now recognised as a crucial element of the success of a production technology (Parekh et al, 1989; Routier et al, 1997; Morrow, 2001; Raju et al, 2000, 2001). The main cell types used in cell culture processes are either hamster or mouse-derived, so do not produce the same sugar pattern on proteins as human cells (Scrip, Jun. 8th 2001). Mammalian and particularly plant transgenic systems produce different types of post-translational modifications on expressed proteins. The sugar profile is crucially important to the manner in which the human immune system reacts to the protein. Raju et al, (2000) found that glycosylated chicken proteins have a sugar profile that is more similar to that of glycosylated human proteins than non-human mammalian proteins, which should be a significant advantage in developing a therapeutic product.

It can therefore be seen that the avian egg, particularly from the chicken, offers several major advantages over cell culture as a means of production and the other transgenic production systems based upon mammals or plants. Direct application of the methods used in the production of transgenic mammals to the genetic manipulation of birds has not been possible because of specific features of the reproductive system of the laying hen. Following either natural or artificial insemination, hens will lay fertile eggs for approximately 10 days. They ovulate once per day, and fertilisation occurs almost immediately, while the ovum is at the top of the oviduct. The egg spends the next 20-24 hours in the oviduct, where the albumen (egg white) is laid down around the yolk, plumping fluid is added to the albumen and finally the shell membranes and the shell itself are laid down. During this time, cell division is rapid, such that by the time the egg is laid, the embryo comprises a blastoderm, a disc of approximately 60,000 relatively undifferentiated cells, lying on the yolk.

The complexities of egg formation make the earliest stages of chick-embryo development relatively inaccessible. Methods employed to access earlier stage embryos usually involve sacrificing the donor hen to obtain the embryo or direct injection into the oviduct. Methods for the production of transgenic mammals have focused almost exclusively on the microinjection of a fertilised egg, whereby a pronucleus is microinjected in vitro with DNA and the manipulated eggs are transferred to a surrogate mother for development to term, this method is not feasible in hens. Four general methods for the creation of transgenic avians have been developed. A method for the production of transgenic chickens using DNA microinjection into the cytoplasm of the germinal disk was developed. The chick zygotes are removed from the oviduct of laying hens before the first cleavage division, transferred to surrogate shells, manipulated and cultured through to hatch (Perry, 1988; Roslin U.S. Pat. No. 5,011,780 and EP0295964). Love et al, (1994) analysed the embryos that survived for at least 12 days in culture and showed that approximately half of the embryos contained plasmid DNA, with 6% at a level equivalent to one copy per cell. Seven chicks, 5.5% of the total number of ova injected, survived to sexual maturity. One of these, a cockerel identified as a potential mosaic transgenic bird, transmitted the transgene to 3.4% of his offspring. These birds have been bred to show stable transmission of the transgene. As in transgenic mice generated by pro-nuclear injection, integration of the plasmid DNA is apparently a random event. However, direct DNA microinjection into eggs results in low efficiencies of transgene integration (Sang & Perry, 1989). It has been estimated that only 1% of microinjected ova give rise to transgenic embryos and of these 10% survive to hatch. The efficiency of this method could be improved by increasing the survival rate of the cultured embryos and the frequency of chromosomal integration of the injected DNA.

A second method involves the transfection of primordial germ cells in vitro and transplantation into a suitably prepared recipient. Successful transfer of primordial germ cells has been achieved, resulting in production of viable gametes from the transferred germ cells. Transgenic offspring, as a result of gene transfer to the primordial germ cells before transfer, have not yet been described.

The third method involves the use of gene transfer vectors derived from oncogenic retroviruses. The early vectors were replication competent (Salter, 1993) but replication defective vectors have been developed (see, eg. U.S. Pat. No. 5,162,215 and WO 97/47739). These systems use either the reticuloendotheliosis virus type A (REV-A) or avian leukosis virus (ALV). The efficiency of these vectors, in terms of production of founder transgenic birds; is low and inheritance of the vector from these founders is also inefficient (Harvey et al, 2002). These vectors may also be affected by silencing of expression of the transgenes they carry as reports suggest that protein expression levels are low (Harvey et al, 2002).

The fourth method involves the culture of chick embryo cells in vitro followed by production of chimeric birds by introduction of these cultured cells into recipient embryos (Pain et al, 1996). The embryo cells may be genetically modified in vitro before chimera production, resulting in chimeric transgenic birds. No reports of germline transmission from genetically modified cells are available.

Although much work has been carried out on retroviral vectors derived from viruses such as ALV and REV as mentioned previously, the limitations of such vectors have prevented more widespread application. Much of the research and development of viral vectors was based on their use in gene therapy applications and so resulted in the demonstration that vectors based on lentiviruses were able to infect nondividing cells, a clear advantage in clinical gene therapy applications. Lentiviruses are a subgroup of the retroviruses which include a variety of primate viruses eg. human immunodeficiency viruses HIV-1 and 2 and simian immunodeficiency viruses (SIV) and non-primate viruses (eg. maedi-visna virus (MVV), feline immunodeficiency virus (FIV), equine infectious anemia virus (EIAV), caprine arthrithis encephalitis virus (CAEV) and bovine immunodeficiency virus (BIV). These viruses are of particular interest in development of gene therapy treatments, since not only do the lentiviruses possess the general retroviral characteristics of irreversible integration into the host cell DNA, but as mentioned previously, also have the ability to infect non-proliferating cells. The dependence of other types of retroviruses on the cell proliferation status has somewhat limited their use as gene transfer vehicles. The biology of lentiviral infection can be reviewed in Coffin et al, (1997) and Sanjay et al, (1996).

An important consideration in the design of a viral vector is the ability to be able to stably integrate into the genome of cells. Previous work has shown that oncoretroviral vectors used as gene transfer vehicles have had somewhat limited success due to the gene silencing effects during development. Jahner et al, (1982) showed that use of the vector based on the Moloney murine leukemia virus (MoMLV) for example, is unsuitable for production of transgenic animals due to silencing of the virus during the developmental phase, leading to very low expression of the transgene. It is therefore essential that any viral vector used for production of transgenic birds does not exhibit gene silencing. The work of Pfeifer et al, (2002) and Lois et al, (2002) on mice has shown that a lentiviral vector based on HIV-1 is not silenced during development.

The bulk of the developmental work on lentiviral vectors has been focused upon HIV-1 systems, largely due to the fact that HIV, by virtue of its pathogenicity in humans, is the most fully characterised of the lentiviruses. Such vectors tend to be engineered as to be replication incompetent, through removal of the regulatory and accessory genes, which render them unable to replicate. The most advanced of these vectors have been minimised to such a degree that almost all of the regulatory genes and all of the accessory genes have been removed.

The lentiviral group have many similar characteristics, such as a similar genome organisation, a similar replication cycle and the ability to infect mature macrophages (Clements & Payne, 1994). One such lentivirus is Equine Infectious Anemia Virus (EIAV). Compared with the other viruses of the lentiviral group, EIAV has a relatively simple genome: in addition to the retroviral gag, pol and env genes, the genome only consists of three regulatory/accessory genes (tat, rev and S2). The development of a safe and efficient lentiviral vector system will be dependent on the design of the vector itself. It is important to minimise the viral components of the vector, whilst still retaining its transducing vector function. A vector system derived from EIAV has been shown to transduce dividing and non-dividing cells with similar efficiencies to HIV-based vectors (Mitrophanous et al, 1999). Oncoretroviral and lentiviral vectors systems may be modified to broaden the range of tranducible cell types and species. This is achieved by substituting the envelope glycoprotein of the virus with other virus envelope proteins. These include the use of the amphotropic MLV envelope glycoprotein (Page et al, 1990), the baculovirus GP64 envelope glycoprotein (Kumar et al, 2003), the adenovirus AD5 fiber protein (Von Seggern et al, 2000) rabies G-envelope glycoprotein (Mazarakis et al, 2001) or the vesicular stomatitis virus G-protein (VSV-G) (Yee et al, 1994). The use of VSV-G pseudotyping also results in greater stability of the virus particles and enables production of virus at higher titres.

It is an aim of the present invention to provide an efficient method for transferring a transgene construct to avian embryonic cells so as to create a transgenic bird which expresses the gene in its tissues, especially, but not exclusively, in the cells lining the oviduct so that the translated protein becomes incorporated into the produced eggs.

It is also an aim of the present invention to provide a vehicle and a method for transferring a gene to avian embryonic cells so as to create a transgenic bird which has stably incorporated the transgene into a proportion or all of its germ cells, resulting in transmission of the transgene to a proportion of the offspring of the transgenic bird. This germ line transmission will result in a proportion of the offspring of the founder bird exhibiting the altered genotype.

It is a further aim of the present invention to provide an efficient method for genetic modification of avians, enabling production of germ line transgenic birds at high frequency and reliable expression of transgenes.

According to the present invention there is provided a method for the production of transgenic avians, the method comprising the step of using a lentivirus vector system to deliver exogenous genetic material to avian embryonic cells or cells of the testes.

The lentivirus vector system includes a lentivirus transgene construct in a form which is capable of being delivered to and integrated with the genome of avian embryonic cells or cells of the testes.

Preferably the lentivirus vector system is delivered to and integrated at an early stage of development such as early cleavage when there have only been a few cell divisions.

In one embodiment the lentivirus transgene construct is injected into the subgerminal cavity of the contents of an opened egg which is then allowed to develop.

The Perry Culture system of surrogate shells may be used.

Alternatively methods used by Bosselmann et al. or Speksnijder and Ivarie of windowing of the egg can be used. In these methods an embryo in a newly laid egg may be accessed by cutting a window in the egg shell and injecting the lentivirus vector system into the embryonic subgerminal cavity. The egg may then be sealed and incubated.

In another embodiment the construct is injected directly into the sub-blastodermal cavity of an egg.

Typically the genetic material encodes a protein.

The genetic material may encode for any of a large number of proteins having a variety of uses including therapeutic and diagnostic applications for human and/or veterinary purposes and may include sequences encoding antibodies, antibody fragments, antibody derivatives, single chain antibody fragments, fusion proteins, peptides, cytokines, chemokines, hormones, growth factors or any recombinant protein.

The invention thus provides a transgenic avian.

Preferably the transgenic avian produced by the method of the invention has the genetic material incorporated into at least a proportion of germ cells such that the genetic material will be transmitted to at least a proportion of the offspring of the transgenic avian.

The invention also provides the use of a lentivirus vector system in the production of a transgenic avian.

It has been surprisingly observed that the use of lentiviral transgene constructs described by the present invention transduce germ cells of avian embryos with unexpectedly high efficiency. Resulting avians subsequently transmit the integrated vector to a high proportion of offspring and the transgene carried by the vector may be expressed at relatively high levels.

The invention thus provides further transgenic avians.

According to the present invention there is also provided a method for production of an heterologous protein in avians, the method comprising the step of delivering genetic material encoding the protein within a lentivirus vector construct to avian embryonic cells so as to create a transgenic avaian which expresses the genetic material in its tissues.

Preferably the transgenic avian expresses the gene in the oviduct so that the translated protein becomes incorporated into eggs.

The protein can then be isolated from eggs by known methods.

The invention provides the use of a lentivirus construct for the production of transgenic avians.

The invention also provides the use of a lentivirus vector construct for the production of proteins in transgenic avians.

Preferably the lentivirus vector construct is used for the expression of heterologous proteins in specific tissues, preferably egg white or yolk.

The lentivirus as used in this application may be any lentiviral vector but is preferably chosen from the group consisting of EIAV, HIV, SIV, BIV and FIV.

A particularly preferred vector is EIAV.

Any commercially available lentivirus vector may be suitable to be used as a basis for a construct to deliver exogeneous genetic material.

Preferably the construct includes suitable enhancer promoter elements for subsequent production of protein.

A specific promoter may be used with a lentiviral vector construct to result in tissue specific expression of the DNA coding sequence. This may include promoters such as CMV, pCAGGS or any promoter based upon a protein usually expressed in an avian egg; such as ovalbumin, lysozyme, ovotransferrin, ovomucoid, ovostatin, riboflavin-binding protein or avidin.

Preferably the vector construct particles are packaged using a commercially available packaging system to produce vector with an envelope, typically a VSV-G envelope.

Typically the vector may be based on EIAV available from ATCC under accession number VR-778 or other commercially available vectors.

Commercial lentivirus-based vectors for use in the methods of the invention are capable of infecting a wide range of species without producing any live virus and do not cause cellular or tissue toxicity.

The methods of the present invention can be used to generate any transgenic avian, including but not limited to chickens, turkeys, ducks, quail, geese, ostriches, pheasants, peafowl, guinea fowl, pigeons, swans, bantams and penguins.

These lentivirus-based vector systems also have a large transgene capacity which are capable of carrying larger protein encoding constructs such as antibody encoding constructs.

A preferred lentiviral vector system is the LentiVector® system of Oxford BioMedica.

The invention further provides a method to determine the likelihood of expression of a protein in vivo, the method comprising the step of measuring expression of the protein in avian oviduct cells in vitro.

The invention therefore provides the use of avian cells in vitro to determine the likelihood of expression in vivo.

The invention is exemplified with reference to the following non-limiting experiments and with reference to the accompanying drawings wherein:

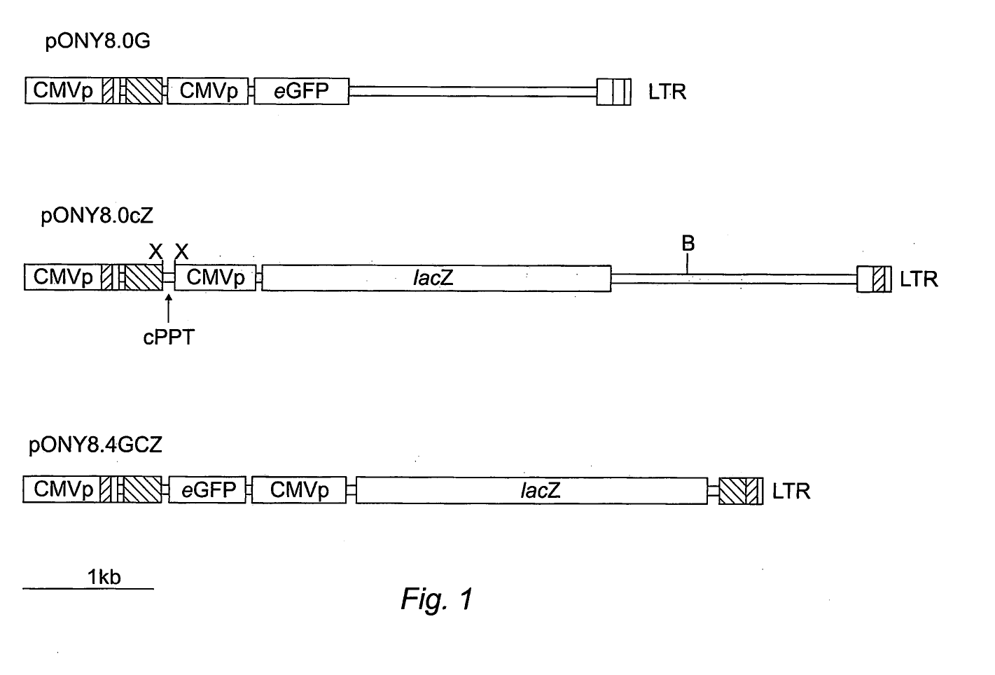

FIG. 1 illustrates a schematic representation of the EIAV vectors used in this study.

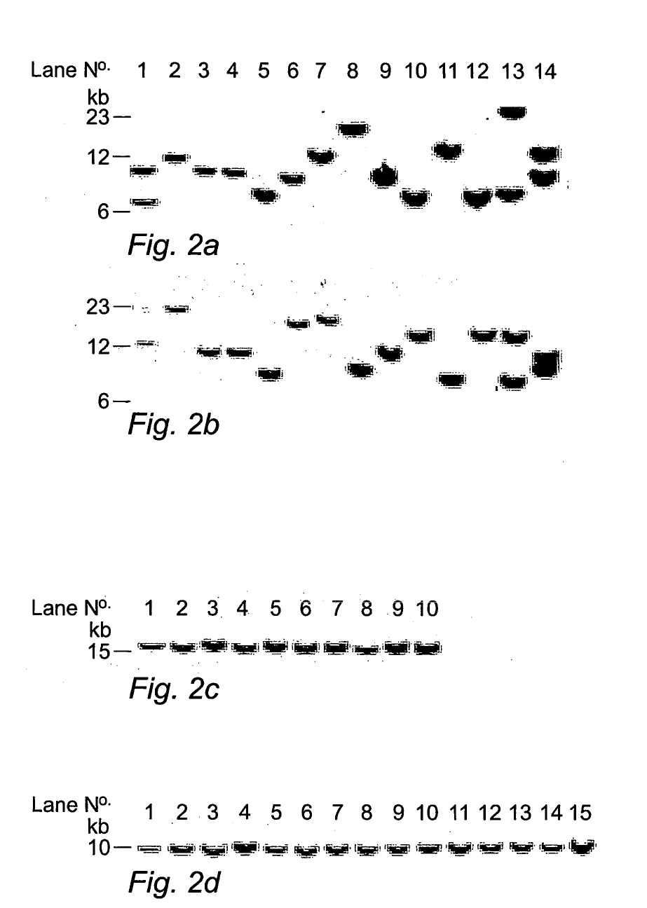

FIG. 2 illustrates Southern transfer analysis of genomic DNA from individual birds to identify proviral insertions.

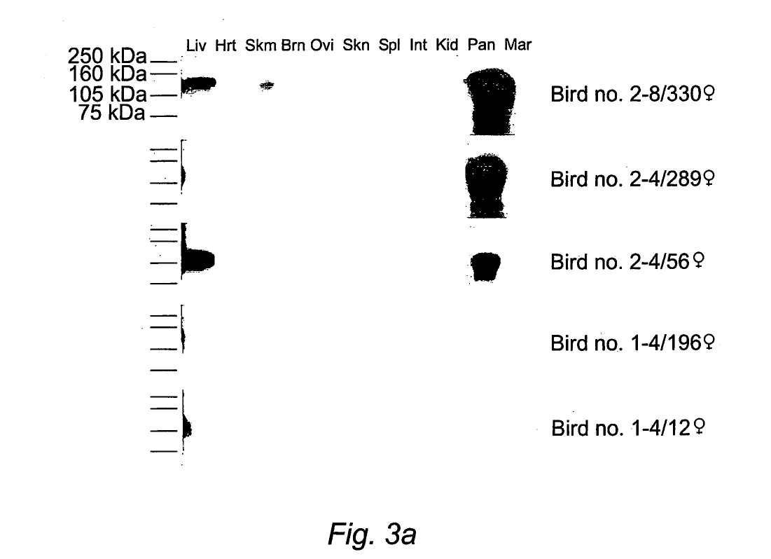

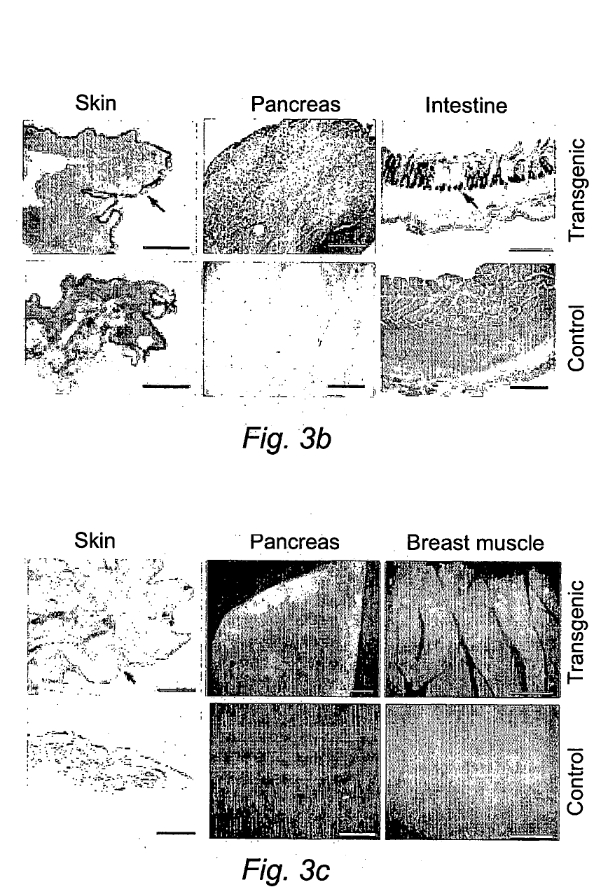

FIG. 3 illustrates reporter gene expression in pONY8.0cZ and pONY8.0G G₁ transgenic birds.

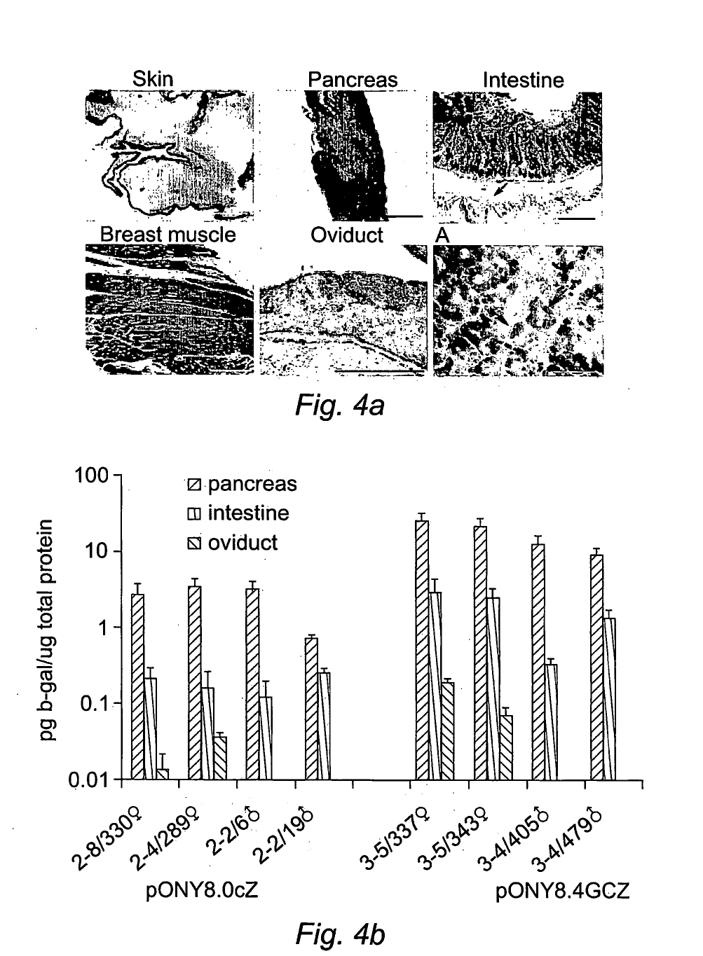

FIG. 4 illustrates reporter gene expression in pONY8.4GCZ Gᵢ transgenic birds.

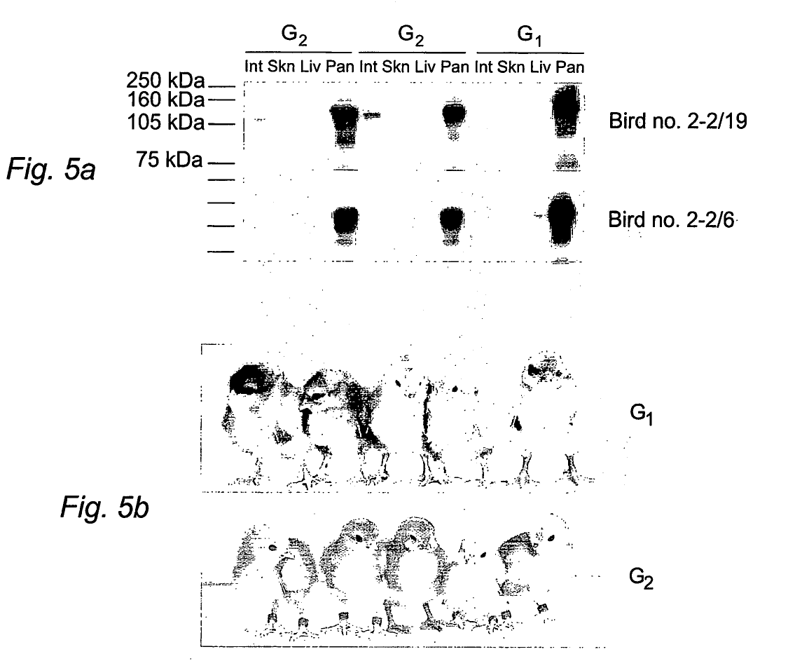

FIG. 5 illustrates reporter gene expression in G₂ transgenic birds.

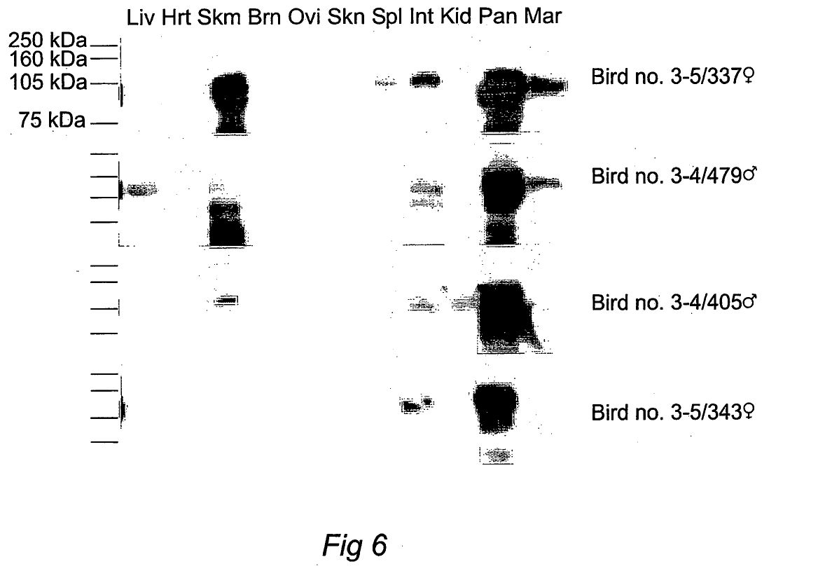

FIG. 6 illustrates Western analysis of pONY8.4GCZ G₁ birds.

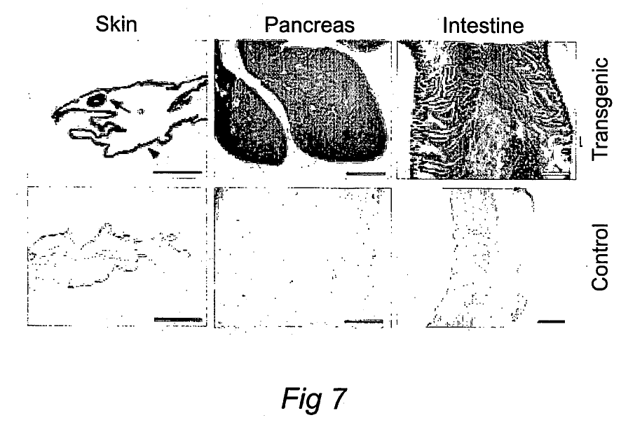

FIG. 7 illustrates reporter gene expression in pONY8.0cZ G₂ birds.

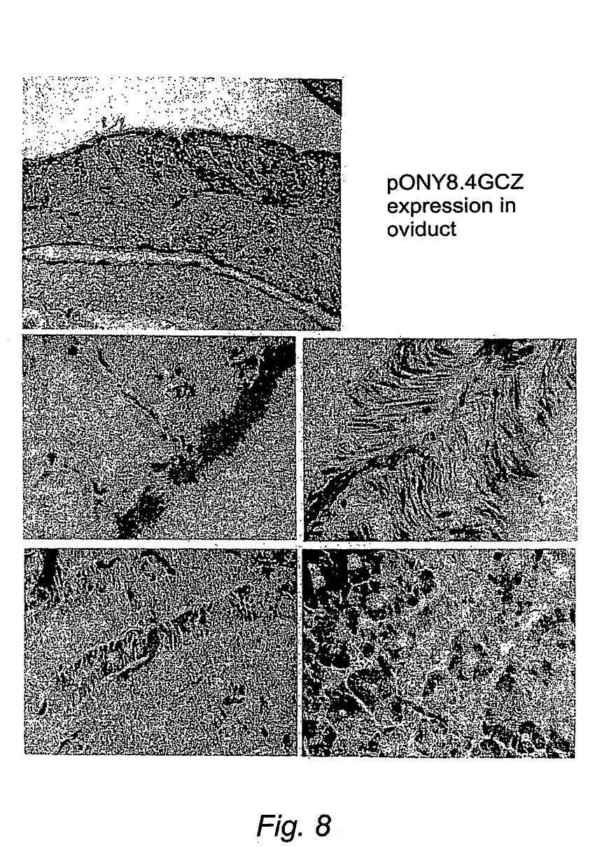

FIG. 8 illustrates lacZ expression in the oviduct of a transgenic bird.

### EXPERIMENT 1

Freshly laid, fertile hen's eggs were obtained which contain developing chick embryos at developmental stages X-XIII (Eyal-Giladi & Kochav, 1976). An egg was opened, the contents transferred to a dish and 2-3 microlitres of a suspension of lentiviral vector virus particles was injected into the subgerminal cavity, below the developing embryo but above the yellow yolk. The vector used was derived from Equine Infectious Anaemia Virus (EIAV) and carried a reporter gene, β-galactosidase (lacZ), under the control of the CMV (cytomegalovirus) enhancer/promoter. The packaging system used to generate the vector viral particles resulted in production of the vector with a VSV-G envelope. The estimated concentration of viral transducing particles was between 5×10⁷ and 1×10⁹ per ml. The embryos were allowed to develop by culturing them using the second and third phases of the Perry culture system (Perry, 1988). 12 embryos were removed and analysed for expression of lacZ after 2 days of incubation and 12 embryos after 3 days of incubation. The embryos and surrounding membranes were dissected free of yolk, fixed and stained to detect expression of the lacZ reporter gene. All embryos showed expression of lacZ in some cells of the embryo and surrounding membranes. The expression was highest in the developing extraembryonic membrane close to the embryo and was limited to a small number of cells in the embryos analysed. These results indicated that all the embryos had been successfully transduced by the injected lentiviral vector.

### EXPERIMENT 2

In a further experiment 40 laid eggs were injected each with 2-3 microlitres of a suspension of the EIAV vector at a titre of 5×10⁸ per ml., into the sub-blastodermal cavity. 13 chicks hatched (33%) and were screened to identify transgenic offspring carrying the lentiviral vector sequence. Samples of the remaining extraembryonic membrane were recovered from individual chicks after hatch, genomic DNA extracted and the DNA analysed by PCR using primers specific to the lentiviral vector sequence. The screen identified 11 chicks as transgenic (85%). The vector sequence was detected in the extraembryonic membrane at a copy number of between 0.4% and 31%, indicating that the chicks were mosaic for integration of the vector. This result was predicted as the embryos were injected with the vector at a stage at which they consisted of at least 60,000 cells. It is unlikely that all the cells in the embryo would be transduced by the viral vector, resulting in chicks that were chimeric for integration of the vector. The 11 chicks were raised to sexual maturity and 7 found to be males. Semen samples were obtained from the cockerels when they reached 16-20 weeks of age. DNA from these samples was screened by PCR and the seven cockerels found to have lentiviral vector sequence in the semen at levels estimated as between 0.1% and 80%. The majority of the samples contained vector sequence at a level above 10%. This suggested that at least 10% of the offspring of these cockerels will be transgenic. Semen was collected from one cockerel, code no. LEN5-20, that had been estimated to have a copy number of the viral vector in DNA from a blood sample as 6%. The copy number estimated from the semen sample was 80%. The semen was used to inseminate stock hens, and the fertile eggs collected and incubated. 9 embryos were recovered after 3 days of incubation, screened by PCR to identify transgenic embryos and stained for expression of the lacZ reporter gene. 3 of the 9 embryos were transgenic and all 3 expressed lacZ but at a very low level in a small number of cells. 12 embryos were recovered after 10 days of incubation and screened as above. 6 embryos were identified as transgenic and lacZ expression detected in 4. The expression was high in several tissues in one embryo and lower in the other 3. These results indicate that 43% of the offspring of cockerel LEN5-20 were transgenic. The expression of the reporter construct carried by the lentiviral vector varied between individual transgenic chicks. It is likely that the individual chicks had copies of the vector genome integrated at different chromosomal sites, which may affect the expression of the transgene. It is also possible that some chicks carried more than one copy of the transgene.

The results outlined here demonstrate that a specific EIAV-derived lentiviral vector, pseudotyped with the VSV envelope protein, can transduce the germ cells of chick embryos with very high efficiency. The resulting birds then transmit the integrated vector to a high proportion of their offspring. The transgene carried by the vector may be expressed to give a functional protein at relatively high levels. The transgene carried by the vector may be designed to express foreign proteins at high levels in specific tissues.

The lentiviral vector may be introduced into the chick at different developmental stages, using modifications of the method described in the example above.

The viral suspension may be injected above the blastoderm embryo in a new laid egg

The viral suspension may be injected into the newly fertilised egg or the early cleavage stages, up to stagex (Eyal-Giladi & Kochav, 1976), by utilizing the culture method of Perry (1988) or recovering eggs from the oviduct and then returning them to a recipient hen by ovum transfer.

The viral suspension may be injected above or below the blastoderm embryo in a freshly laid egg which has been accessed by cutting a window in the shell. The window may be resealed and the egg incubated to hatch (Bosselman et al, 1989).

The viral suspension may be injected into the testes of cockerels and semen screened to detect transduction of the spermatogonia and consequent development of transgenic sperm.

### EXPERIMENT 3

### MATERIAL AND METHODS

EIAV vectors and preparation of virus stocks The vectors pONY8.0cZ and pONY8.0G have been described previously (Pfeifer et al, 2002). The vector pONY8.4GCZ has a number of modifications including alteration of all ATG sequences in the gag-derived region to ATTG, to allow expression of eGFP downstream of the 5′LTR. The 3′ U3 region has been modified to include the Moloney leukaemia virus U3 region. Vector stocks were generated by FuGENE6 (Roche, Lewes, U.K.) transfection of HEK 293T cells plated on 10 cm dishes with 2 μg of vector plasmid, 2 μg of gag/pol plasmid (pONY3.1) and 1 μg of VSV-G plasmid (pRV67) (Lois et al, 2002). 36-48 hours after transfection supernatants were filtered (0.22 μm) and stored at −70° C. Concentrated vector preparations were made by initial low speed centrifugation at 6,000×g for 16 hours at 4° C. followed by ultracentrifugation at 50,500×g for 90 minutes at 4° C. The virus was resuspended in formulation buffer (Lois et al, 2002) for 2-4 hours, aliquoted and stored at −80° C.

Production and analysis of transgenic birds Approximately 1-2 μl of viral suspension was microinjected into the sub-germinal cavity beneath the blastodermal embryo of new-laid eggs. Embryos were incubated to hatch using phases II and III of the surrogate shell ex vivo culture system (Challita & Kohn, 1994). DNA was extracted from the CAM of embryos that died in culture at or after more than twelve days of development using Puregene genomic DNA purification kit (Flowgen, Asby de la Zouche, U.K.). Genomic DNA samples were obtained from CAM of chicks at hatch, blood samples from older birds and semen from mature cockerels. PCR analysis was carried out on 50 ng DNA samples for the presence of proviral sequence. To estimate copy number control PCR reactions were carried out in parallel on 50 ng aliquots of chicken genomic DNA with vector plasmid DNA added in quantities equivalent to that of a single copy gene (1×), a 10-fold dilution (0.1×) and a 100-fold dilution (0.01×) as described previously (Perry, 1988). Primers used:

5′CGAGATCCTACAGTTGGCGCCCGAACAG3′ and 5′ACCAGTAGTTAATTTCTGAGACCCTTGTA-3′. The number of proviral insertions in individual G₁ birds was analysed by Southern transfer. Genomic DNA extracted from whole blood was digested with XbaI or BamHI. Digested DNA was resolved on a 0.6% (w/v) agarose gel then transferred to nylon membrane (Hybond-N, Amersham Pharmacia Biotech, Amersham U.K.). Membranes were hybridised with ³²P-labelled probes for the reporter gene lacZ or eGFP at 65° C. Hybridisation was detected by autoradiography. All experiments, animal breeding and care procedures were carried out under license from the U.K. Home Office.

Expression Analysis

Adult tissues were isolated and fixed for 30 min in 4% paraformaldehyde, 0.25% gluteraldehyde, in phosphate buffered saline (PBS). Tissues were cryo-embedded embedded and sectioned at 14 μm. β-galactosidase activity was detected by incubating at 37° C. in 5 mM potassium ferricyanide, 5 mM potassium ferrocyanide, 2 mM MgCl₂, 0.5 mg/ml X-gal for 90 min (sections) or 4 hours (embryos). GFP images of hatchlings were captured using Fujifilm digital camera (Nikon 60 mm lens) shot through a GFsP-S lens system (BLS, Ltd, Czech Republic). Selected tissues were snap-frozen and total protein was extracted by homogenization in PBS containing protease inhibitors (complete mini, Roche, Lewes, U.K.). Protein concentration was determined by Bradford assay. Either 50 μg (FIG. 4) or 100 μg (FIG. 3) of protein extract was resolved on 12% polyacrylamide gels (Invitrogen, Paisley, U.K.) and transferred to PDVF membranes. Membranes were incubated with mouse anti-β-galactosidase antibody (Promega, Southampton, U.K.) at 1:5000 dilution and donkey anti-mouse IgG-HRP antibody (Santa Cruz Biotech) at 1:2000 dilution and visualized with the ECL western blotting detection system (Amersham Biosciences, Amersham, U.K.). ELISA was performed using β-gal Elisa kit (Roche, Lewes, U.K.).

### RESULTS

Detailed Figure Legends

FIG. 1. Schematic representation of the EIAV vectors used in this study.

The light grey box represents the EIAV packaging signal, and the diagonal lined box in pONY8.4GCZ the MLV U3 region. Restriction sites (XbaI [X], BstEII [B] utilised for Southern blot analysis are indicated. The reporter gene lacZ was used as a probe (FIG. 2).

FIG. 2. Southern transfer analysis of genomic DNA from individual birds to identify proviral insertions. Genomic DNA samples were digested with XbaI (a, c, d) or BstEII (b) and hybridised with a probe for lacZ. (a, b) Analysis of 14 G1 offspring of G0bird no. 1-4 (Table 1) revealed multiple proviral insertions in the G1 birds. (c) Analysis of G1 bird no. 2-2/19 (lane 1) and 14 of his G2 offspring (lanes 2-15) and (d) G1 bird 2-2/6 (lane 1) and 9 of his G2 offspring (lanes 2-10), demonstrated stability of the proviral insertions after germ line transmission.

FIG. 3. Reporter gene expression in pONY8.0cZ and pONY8.0G G₁ transgenic birds.

a Western blot analysis of liver, heart, skeletal muscle, brain, oviduct, skin, spleen, intestine, kidney, pancreas and bone marrow protein extracts from 5 adult G₁ birds each containing single, independent insertions of pONY8.0cZ.100 μg of protein was loaded per lane and β-galactosidase protein detected as described in Experimental Protocols. b Sections of skin, pancreas, and intestine from G₁ 2-2/19 stained for β-galactosidase activity and comparable sections of a non-transgenic control bird (arrowheads indicate epidermis of skin, villi of intestine). Bar=0.5 mm. c Sections of breast muscle, pancreas, and skin from a single copy transgenic or a wildtype bird were visualized for GFP fluorescence (arrowhead indicates epidermis of skin). Bar=0.5 mm.

FIG. 4. Reporter gene expression in pONY8.4GCZ G₁ transgenic birds.

a Sections of tissues from a single copy G₁ bird was stained for β-galactosidase activity (arrow indicates smooth muscle of intestine). Bar=0.5 mm. Panel A: higher magnification of oviduct section. Arrows identify cells lining tubular glands cut in cross-section. Bar=0.05 mm. b Levels of β-galactosidase protein were determined for pONY8.0cZ and pONY8.4GCZ lines. Data points were generated from three independent experiments.

FIG. 5. Reporter gene expression in G₂ transgenic birds.

a Western analysis of protein extracted from intestine, skin, liver and pancreas of G₁ cockerels 2-2/19 and 2-2/6 and two G₂ offspring of each bird. b Top panel: five G₁ offspring of bird ID 4-1. The 4 birds on the left are transgenic for pONY8.0G and express eGFP. The bird on the right is not transgenic. Bottom panel: five G₂ offspring of bird ID 4-1/66. The bird in the center is not transgenic.

FIG. 6. Western analysis of pONY8.4GCZ G1 birds. Western blot analysis of liver, heart, skeletal muscle, brain, oviduct, skin, spleen, intestine, kidney, pancreas and bone marrow protein extracts from 4 adult G₁ birds each containing single, independent insertions of pONY8.4GCZ. 100 μg of protein was loaded per lane and β-galactosidase protein detected as described in Experimental Protocols.

FIG. 7. Reporter gene expression in pONY8.0cZ G2 transgenic birds.

Sections of skin, pancreas and intestine (arrowhead indicates epidermis, arrow indicates feather follicle) from a G₂ offspring of 2-2/19 stained for β-galactosidase activity and comparable sections of a non-transgenic control bird. Bar=0.5 mm

Production of G₀ transgenic birds Three different self-inactivating EIAV vectors (FIG. 1) were used, pseudotyped with vesicular stomatitis virus glycoprotein (VSV-G). These vectors have previously been used to transduce a number of tissues in several animal model systems, both in vitro and in vivo (Pfeifer et al, 2002; Rholl et al, 2002; Corcoran et al, 2002; Azzouz et al, 2002). The pONY8.4 vector was modified from pONY8.0 by substitution of Moloney murine leukaemia virus (MoMLV) sequence in the 5′ LTR and deletion of the majority of the viral env gene. The vector preparations were concentrated to give titres of approximately 10⁷ to 10¹⁰ transducing units per millilitre (T.U./ml). One to two microlitres of concentrated vector was injected into the subgerminal cavity below the developing embryonic disc of new-laid eggs, which were then cultured to hatch. Genomic DNA was extracted from chorioallantoic membrane (CAM) of hatched G₀ chicks and analysed by PCR to detect the EIAV packaging site sequence. The approximate copy number of the vector with respect to the amount of genomic DNA present was estimated, with a range from the equivalent of one copy per genome to 0.01 copies per genome (see Experimental Protocol). All chicks were raised to sexual maturity and genomic DNA from semen samples from males was similarly screened by PCR.

Four experiments were carried out. The virus pONY8.0cZ was injected at a titre of 5×10⁷ T.U./ml in experiment 3.1 and 5×10⁸ T.U./ml in experiment 3.2. In experiment 3.3 the virus pONY8.4GCZ was injected at a concentration of 7.2×10⁸ T.U./ml and in experiment 3.4 pONY8.0G was used at 9.9×10⁹ T.U./ml. A total of 73 eggs were injected in the four experiments from which 20 (27%) chicks hatched. The results of the PCR screen of hatched male and female chicks from each experiment are shown in Table 1. Fourteen of the twenty G₀ birds contained vector sequences at levels estimated to be between 0.5 to 0.01 copies per genome equivalent. The vector pONY8.0cZ transduced the chick embryos more efficiently than the vector pONY8.4GCZ when injected at a similar concentration, possibly due to the presence of the viral cPPT sequence that is involved in nuclear import of the viral DNA genome (Lois et al, 2002). The results also show that transgenic birds can be produced using titres as low as 5×10⁷ T.U./ml, but that transduction frequency increases if higher titres are used.

Germ line transmission from G₀ males Semen samples were collected from the 12 G₀ males when they reached sexual maturity, between 16 and 20 weeks of age. The results of PCR screens of genomic DNA extracted from these samples are given in Table 1. These showed that vector sequences were present in the germ line of all the cockerels, even those that had been scored as not transgenic when screened at hatch. This was confirmed by breeding from 10 of the 12 cockerels by crossing to stock hens and screening their G₁ offspring to identify transgenic birds. All 10 cockerels produced transgenic offspring, with frequencies ranging from 4% to 45%. The frequencies of germ line transmission were very close to those predicted from the PCR analysis of semen DNA but, in every case, higher than predicted from analysis of DNA from CAM samples taken at hatch. Blood samples were taken from several cockerels and PCR analysis closely matched the results from the CAM DNA analysis (data not shown). The results suggest a germ line transduction frequency approximately 10-fold higher than that of somatic tissues.

Analysis of G₁ transgenic birds and transmission to G₂

The founder transgenic birds were transduced at a stage of development when embryos consist of an estimated 60,000 cells, approximately 50 of which are thought to give rise to primordial germ cells (Biene mann et al, 2003; Ginsburg & Eyal-Giladi, 1987). We predicted that the G₁ birds to result from separate transduction events of individual primordial germ cells and that different birds would have independent provirus insertions, representing transduction of single germ cell precursors. It was also possible that individual cells would have more than one proviral insertion. Four G₀ cockerels, transduced with pONY8.0cZ (experiments 3.1 and 3.2), were selected for further analysis of their transgenic offspring (Table 2). Genomic DNA from individual G₁ birds was analysed by Southern blot. Samples were digested separately with XbaI and Bst EII, restriction enzymes that cut within the integrated EIAV provirus but outside the probe region (FIG. 1), and hybridised with probes to identify restriction fragments that would represent the junctions between the proviral insertions and the genomic DNA at integration sites. This enabled estimation of the number of proviral insertions in each G₁ bird and of the number of different insertions present in the offspring of each G₀ analysed. An example of this analysis is shown in FIGS. 2a,b and the results summarised in Table 2. The majority of G₁ birds carried single proviral insertions but several contained multiple copies, with a maximum of 4 detected in one bird. Some offspring of each G₀ bird carried the same proviral insertion, indicating that they were derived from the same germ cell precursor.

Three male G₁ offspring of bird 2-2 (2-2/6,16 and 19) were crossed to stock hens to analyse transmission frequency to the G₂ generation. Cockerels 2-2/6 and 2-2/19 had single proviral insertions and the ratios of transgenic to non-transgenic offspring, 14/30 (47%) and 21/50 (42%), did not differ significantly from the expected Mendelian ratio. Cockerel 2-2/16 had two proviral insertions and 79% (27/34) of the G₂ offspring were transgenic, reflecting the independent transmission of two insertions. Southern transfer analysis was used to compare the proviral insertion present in birds 2-2/6 and 2-2/19 with 9and 14 of their G₂ offspring, respectively (FIGS. 2c,d). Identical restriction fragments were observed in parents and offspring, indicating that the proviruses were stable once integrated into the genome.

Transgene expression in G₁ and G₂ transgenic birds The vectors pONY8.0cZ and pONY8.4GCZ carried the reporter gene lacZ under control of the human cytomegalovirus (CMV) immediate early enhancer/promoter (CMVp) and pONY8.0G carried the reporter eGFP, also controlled by CMVp. Expression of lacZ was analysed by staining of tissue sections to detect β-galactosidase activity and by western analysis of protein extracts from selected tissues isolated from adult birds, to identify β-galactosidase protein. Expression of eGFP was analysed using UV illumination.

Protein extracts were made from a range of tissues from seven pONY8.0cZ G₁ birds, each containing a different single provirus insertion. A protein of the expected 110 kDa was detected in some tissues in each transgenic bird. Expression was consistently high in pancreas and lower levels of protein were present in other tissues, including liver, intestine and skeletal muscle. The analysis of five of these birds is shown in FIG. 3a. β-galactosidase was detected in most tissues on longer exposures of the western blot (data not shown). The pattern of expression was consistent between the individual birds but the overall amounts of protein varied. Sections of tissues from an adult pONY8.0cZ G₁ bird were stained (FIG. 3b). Intense staining was observed throughout the exocrine pancreas and in other tissues, such as the epithelium of the skin and villi of the small intestine. Expression analysis of GFP in sections of tissue from a pONY8.0G bird detected expression in the pancreas, skin and breast muscle (FIG. 3c) and weak expression in the intestine (data not shown). These results show that transgenic birds produced with the same EIAV vector but carrying different reporter genes showed similar patterns of expression.

Western analysis of tissues from six G₁ birds carrying different single proviral insertions of pONY8.4GCZ detected lacZ expression in four birds, in a pattern similar to that seen in the pONY8.0cZ transgenic birds (FIG. 6). However, staining of tissue sections revealed a more extensive pattern of expression than was observed in birds transgenic for pONY8.0cZ. β-galactosidase activity was detected additionally in the smooth muscle of the intestine, blood vessels underlying the epidermis and in tubular gland cells of the oviduct (FIG. 4a). An ELISA assay was used to quantify the differences in levels of expression of β-galactosidase between transgenic birds carrying the pONY8.0 and pONY8.4 vectors (FIG. 4b). β-galactosidase levels were higher in pONY8.4GCZ birds in all tissues assayed than in pONY8.0cZ birds. Levels in pancreatic extracts were approximately 6-fold higher and expression in bird no. 3-5/337 was 30 pg per microgram of tissue, or 3% of total protein.

To establish if transgene expression was maintained after germ line transmission, expression in G₂ birds carrying the vectors pONY8.0cZ and pONY8.0G was examined. Western analysis was carried out on tissue extracts from two G₁ cockerels, 2-2/6 and 2-2/19, that each had a single proviral insertion, and two G₂ offspring from each cockerel. (FIG. 5a). β-galactosidase protein levels are very similar in the parent and two offspring and the patterns of expression, predominantly in the pancreas, are also very similar. Staining of tissue sections from a G₂ bird demonstrated expression patterns comparable to that observed in the parent (FIG. 7). GFP fluorescence was readily detected in live G₁ chicks carrying pONY8.0G and the G₂ offspring of one of these birds showed a similar level of expression (FIG. 5b).

FIG. 8 shows a range of sections from the oviduct of a transgenic hen carrying the vector pONY8.4GCZ carrying the reporter gene lac Z. Blue stain is apparent in the sections illustrating expression of lacZ.

### DISCUSSION

We have demonstrated that the lentiviral vector system that we have tested is a very efficient method for production of germ line transgenic birds. In the experiments described here twelve cockerels were produced after injection of concentrated suspension of viral vector particles immediately below the blastoderm stage embryo in new laid eggs. We bred from ten founder cockerels and all produced transgenic offspring, with frequencies from 4 to 45%. Even the lowest frequency of germ line transmission obtained is practical in terms of breeding to identify several G₁ transgenic birds from one founder cockerel, in order to establish independent lines carrying different proviral insertions. This method of sub-blastodermal injection is very similar to the methods used previously (Salter & Crittenden, 1989; Bosselman et al, 1989; Harvey et al, 2002) to introduce retroviruses into the chicken. The high success rate may be due to a number of factors, including the ability of lentivral vectors to transduce non-dividing cells, the use of the VSV-G pseudotype, that has previously been used to introduce a retroviral vector into quail (Karagenc et al, 1996), and the high titres used compared to previous transgenic studies. The chick embryo in a laid egg is a disc consisting of a single layer of cells, lying on the surface of the yolk, with cells beginning to move through the embryo to form the hypoblast layer below the embryonic disc (Mizuarai et al, 2001). Primordial germ cells also migrate from the embryonic disc, through the subgerminal cavity and on to the hypoblast below. It is possible that during the developmental stages immediately after the virus injection, the primordial germ cells migrate through the suspension of viral particles, thus accounting for the higher frequency of germ cell transduction compared to that of cells of the CAM or blood.

We have shown that the majority of G₁ transgenic birds contain a single proviral insertion but that some birds contain multiple insertions. These results indicate that it will be easy to use this vector system to generate transgenic birds with single vector-transgene insertions and to breed several lines from the same G₀ bird, with the provirus inserted at different chromosomal loci. Levels of expression of a transgene, introduced by a particular vector but integrated at different sites within the chicken genome, are likely to vary. The analysis of transmission from G₁ to G₂ indicates that it will be simple to establish lines carrying stable transgene insertions, using the lentiviral vectors described.

Expression of the reporter gene lacZ was detected in founder (G₀) G₁ and G₂ birds. The expression of lacZ was directed by human CMVp (nucleotides −726 to +78), an enhancer/promoter generally described as functioning ubiquitously in many cell types. This is usually the case if it is used in cell culture transfection experiments but expression in transgenic mice from the CMVp varies between tissues. In particular, it has been reported that CMVp transgene shows predominant expression in exocrine pancreas in transgenic mice (Eyal-Giladi & Kochav, 1976). We have shown that the pattern of expression of both lacZ and GFP in embryos and birds is predominantly in the pancreas, although it is expressed at varying levels in most tissues. Expression from the third generation EIAV vector pONY8.4 was significantly higher than from the pONY8.0 vector, possibly due to increase in mRNA stability in the former resulting from removal of instability elements in the env region. Transgene expression was not detected in a small number of pONY8.4GCZ transgenic birds, possibly due to the inclusion of MoMLV sequence in the vector that may induce silencing (Zhan et al, 2000). The expression pattern seen in G₁ birds is maintained after germ line transmission to G₂. These results indicate that transgene-specific expression, from transgenes introduced using lentiviral vectors, is maintained after germ line transmission, as has been described in the mouse and rat (Naldini et al, 1996). The size of transgenes that can be incorporated in lentiviral vectors is limited and therefore some tissue-specific regulatory sequences may be too big for use in these vectors. The limit has yet to be defined but is likely to be up to 8 kb, as EIAV vectors of 9 kb have been successfully produced (Lois et al, 2002).

Expression of lacZ in the oviduct (FIG. 8) demonstrates that the cells which synthesize egg white proteins can express foreign proteins in transgenic birds carrying an integrated lentiviral vector system encoding a protein.

The study described here is an evaluation of the possible application of lentiviral vectors for the production of transgenic birds. We have shown that we can obtain a very high frequency of germline transgenic birds, stable transmission from one generation to the next, and a pattern of transgene expression that is maintained after germline transmission. These results indicate that the use of lentiviral vectors will overcome many of the problems encountered so far in development of a robust method for production of transgenic birds. The application of this method for transgenic production will allow many transgene constructs to be tested to determine those that express in appropriate tissues and at required levels. Recently an ALV vector has been used to generate a transgenic line in which expression and accumulation in egg white of low amounts of biologically active protein was demonstrated (Rapp et al, 2003). Although the amounts of protein produced, micrograms of protein per egg, is not at a level that will facilitate commercial production, the analysis of the protein purified from egg white supports the aim that transgenic hens may be used as bioreactors. The use of lentiviral vectors may overcome the problems associated with transgene incorporation and expression using oncoretroviral vectors. The development of an efficient method for production of transgenic birds is particularly timely as the chicken genome sequence is due to be completed this year and the value of the chick as a model for analysis of vertebrate gene function is increasing (Mozdziak et al, 2003).

### EXPERIMENT 4

Experiments are being carried out with the Invitrogen ViraPower™ system. The chickenised R24 minibody coding sequence is inserted into the pLenti6/V5 plasmid immediately downstream of the constitutive CMV promoter. ViraPower™ 293FT cells are then cotransfected with the pLenti6/V5/R24 expression construct and the optimised ViraPower™ packaging mix. Finally packaged virus-containing tissue culture supernatant is harvested. One intended use for the Invitrogen ViraPower™ system is as a high efficiency transfection reagent. The presence of the blasticidin resistance gene on the pLenti6/V5 plasmid confers the ability to preferentially select transduced populations. This means relatively low titre viral harvests are adequate. However, for the experimental work described below, more concentrated viral harvests are required. Two methods of viral concentration are being evaluated. First, the use of spin concentration via Centrikon Plus20 spin columns. Second, the use of a standard ultracentrifugation protocol.

The structure of the RNA genome of the concentrated packaged viral vectors is being analysed by both Northern blotting and Reverse Transcriptase-Polymerase Chain Reaction (RT-PCR). Reverse transcription is carried out with several reverse primers, oligo dT, random hexamers and a primer specific to the 3′LTR, to ensure that a representative sample of viral genomes are converted to cDNA. The integrity of the cR24 coding sequence in the cDNA samples is verified using individual PCR reactions optimised to amplify specific sequences.

The packaged pLenti6/V5/R24 viral vector is also being used for transduction of 293T cells in vitro. Multiple pLenti6/V5/R24 viral dilutions are prepared in standard tissue culture medium with the addition of polybrene. The virus/medium/polybrene mixes are then added to cells. After three hours the tissue culture medium is replenished until after a further 72 hrs the medium is harvested. The level of secreted cR24 minibody is then quantified via ELISA. Transduced cells are also selected with blasticidin for a period of 7-10 days before medium is harvested. Here also the level of secreted cR24 minibody is quantified via ELISA.

Furthermore, the packaged pLenti6/V5/R24 viral vector is also being used for the transduction of chick embryos in vivo via injection into the subgerminal cavity, below the developing embryo but above the yellow yolk.

### BIBLIOGRAPHY

Andersson, R. & Mynahan, R. May 2001 ‘The Protein Production Challenge’ In Vivo: The Business and Medicine Report 15 (5) Windhover Information Inc.
Azzouz, M. et al 2002 ‘Multicistronic lentiviral vector-mediated striatal gene transfer of aromatic L-amino acid decarboxylase, tyrosine, hydrolase and GTP cyclohydrolase I induces sustained transgene expression, dopamine production and functional improvement in a rat model of Parkinson's disease’ J. Neurosci. 22 10302-10312.
Bienemann, A. S. et al 2003 ‘Long-term replacement of a mutated non-functional CNS gene: reversal of hypothalamic diabetes insipidus using an EIAV-based lentiviral vector expressing arginine vasopressin’ Mol. Ther. 7 588-596
Biopharm Cost of Goods Analysis: A Comparison between Transgenic and Cell Culture Manufacture. November 2001.
Bosselmann, R. A., Hsu, R. Y., Boggs, T., Hu, S., Bruszewski, J., Ou, S.,.Kozar, L., Martin, F. Green, C, and Jacobsen, F. 1989 ‘Germline transmission of exogenous genes in the chicken’ Science 243 (4890) p 533-535.
Brackett, B. G., Baranska, W., Sawicki, W., Koprowski, H. 1971 ‘Uptake of heterologous genome by mammalian spermatozoa and its transfer to ova through fertilization’. Proc. Natl. Acad. Sci. USA. 68 (2) 353-7.
Challita, P. M. & Kohn, D. B. 1994 ‘Lack of expression from a retroviral vector after transduction of murine haematopoietic stem cells is associated with methylation in vivo’ Proc. Natl. Acad. Sci. USA. 91 2567-2571.
Clements, J. E. and Payne, S. L. 1994 ‘Molecular basis of the pathobiology of lentiviruses’ Virus Research 32 (2) p 97-109.
Coffin, J. M., Hughes, S. M. and Varmus, H. E. 1997 ‘Retroviruses’ Cold Spring Harbor Laboratory Press.
Corcoran et al 2002 ‘Retinoic acid receptor β2 and neurite outgrowth in the adult mouse spinal cord in vitro’ J. Cell Sci. 115 3779-3786
Curtis, H. & Barnes, N. S. 1989 ‘Biology’ 5^{th} Edition Worth Publishers, Inc.
Dove, A. 2000 ‘Milking the genome for profit’ Nature Biotechnology 18 p 1045-1050.
Etches, R. J. & Gibbins, A. M. (Eds) 2000 ‘Manipulation of the Avian Genome’ CRC Press.
Eyal-Giladi, H. and Kochav, S. 1976 ‘From cleavage to primitive streak formation.’ Developmental Biology 49(2) p 321-337.
Gardner, R. L. 1968 ‘Mouse chimeras obtained by the injection of cells into the blastocyst’. Nature 220 (167) p 596-7.
Ginsburg, M. & Eyal-Giladi, H. 1987 ‘Primordial germ cells of the young chick blastoderm originate from the central zone of the A. pellucida irrespective of the embryo-forming process’ Development 101 209-211
Gordon, J. W. & Ruddle, F. H. 1981 ‘Integration and stable germline transmission of genes injected into mouse pronuclei’. Science 214 (4526) p 1244-6.
Harvey, A. J., Speksnijder, G., Baugh, L. R., Morris, J. A. and Ivarie, R. 2002 ‘Expression of exogenous protein in the egg white of transgenic chickens’ Nature Biotechnology 19 p 396-399.
Jaenisch, R. & Mintz, B. 1974 ‘Simian virus 40 DNA sequences in DNA of healthy adult mice derived from preimplantation blastocysts injected with viral DNA’. Proc. Natl. Acad. Sci. USA. 71 (4) 1250-4.
Jahner, D., Stuhlmann, H., Stewart, C. L., Harbers, K., Lohler, J., Simon, I. and Jaenisch, R. 1982 ‘De novo methylation and expression of retroviral genomes during mouse embryogenesis’ Nature 298 (5875) p 623-628.
Karagenc, L., Cinnamon, Y., Ginsburg, M. & Petitte, J. 1996 ‘Origin of primordial germ cells in the prestreak chick embryo’ Dev. Genet. 19 290-301
Kumar, M., Bradow, B. P. and Zimmerberg, J. 2003 ‘Large-scale production of pseudotyped lentiviral vectors using baculovirus GP64’ Human Gene Therapy 14 67-77
Lois, C., Hong, E. J., Pease, S., Brown, E. J. and Baltimore, D. 2002 ‘Germline Transmission and Tissue-specific Expression of Transgenes delivered by Lentiviral Vectors’ Science 295 868-872.
Love, J., Gribbin, C., Mather, C and Sang, H. 1994 ‘Transgenic Birds by Microinjection’ Biotechnology 12.
Mazarakis, N. D., Azzouz, M., Rohll, J. B., Ellard, F. M., Wilkes, F. J., Olsen, A. L., Carter, E. E., Barber, R. D., Baban, D. F., Kingsman, S. M., Kingsman, A. J., O'Malley, K and Mitrophanous, K. A. 2001 ‘Rabies virus glycoprotein pseudotyping of lentiviral vectors enables retrograde axonal transport and access to the nervous system after peripheral delivery’ Hum. Mol. Genet. 10(19) 2109-2021
Mitrophanous, K. A., Yoon, S., Rohl, J. B., Patil, D., Wilkes, F. J., Kim, V. N., Kingsman, S. M., Kingsman, A. K. and Mazarakis, N. D. 1996 ‘Stable Gene Transfer to the Nervous System using a Non-Primate Lentiviral Vector’ Gene Therapy 6 p1808-1818.
Mizuarai, S., Ono, K., Yamaguchi, K., Nishijima, K., Kamihira, M. & Iijima, S., 2001 ‘Production of transgenic quails with high frequency of germ-line transmission using VSV-G pseudotyped retroviral vector’ Biochem. Biophys. Res. commun. 286 456-463
Mozdziak, P. E., Borwornpinyo, S., McCoy, D. W., Petitte, J. N. 2003 ‘Development of transgenic chickens expressing bacterial β-galactosidase’ Dev. Dyn. 226 439-445

Morrow, K. J. Jr. 2001 ‘Antibody Production: Planning Well Ahead to Meet Future Demand’ Genetic Engineering News 21(7) April 1.
Mozdziak, P. E., Borwornpinyo, S., McCoy, D. W. & Petitte, J. N. 2003 ‘Development of transgenic chickens expresing bacterial
Naldini, L., Blomer, U., Gallay, P., Ory, D., Mulligan, R., Gage, F. H. & Verma Trono, D. 1996 ‘In vivo gene delivery and stable transduction of nondividing cells by a lentiviral vector’ Science 272 263-267
Pain, B., Clark, E. M., Shen, M., Nakazawa, H., Sakurai, M., Samarut, J. and Etches, R. J. 1996 ‘Long-term in vitro culture and characterisation of avian embryonic stem cells with multiple morphogenetic potentialities’ Development 122 p 2339-2348.
Parekh, R. B., Dwek, R. A., Rudd,.P. M., Thomas, J. R., Rademacher, T. W., Warren, T., Wun, T-C., Hebert, B., Reitz, B., Palmier, M., Ramabhadran, T & Tierneier, D. C. 1989 ‘N-Glycosylation and In vitro Enzymatic Activity of Human Recombinant Tissue Plasminogen Activator Expressed in Chinese Hamster Ovary Cells and a Murine Cell-Line’ Biochemistry 28 7670-7679
Perry, M. M. 1988 ‘A complete culture system for the chick embryo’ Nature 331 (6151) p 70-72.
Pfeifer, A., Ikawa, M., Dayn, Y. and Verma, I. M. 2002 ‘Transgenesis by Lentiviral vectors: Lack of gene silencing in mammalian embryonic stem cells and preimplantation embryos’ Proceedings of the National Academy of Sciences 99(4) p 2140-2145.
Raju, T., Briggs, J., Borge, S., Jones, A. 2000 ‘Species-specific variation in Glycosylation of IgG: evidence for the species-specific sialylation and brand-specific galactosylation and importance for engineering recombinant glycoprotein therapies’. Glycobiology 10 477-486.
Raju, T., Briggs, J., Chamow, S. M., Winkler, M. E. & Jones, A. J. S. 2001 ‘Glycoengineering of Therapeutic Glycoproteins: In vitro Galactosylation and Sialylation of Glycoproteins with Terminal N-Acetylglucosamine and Galactose residues’ Biochemistry 40 8868-887.6
Rapp,. J. C., Harvey, A. J., Speksnijder, G. L., Hu, W. & Ivarie, R. 2003 ‘Biologically active human interferon α-2b produced in the egg white of transgenic hens’ Transgenic Res. 12 569-575
Rholl, J. B. et al 2002 ‘Design, production, safety, evaluation and clinical applications of non-primate lentiviral vectors’ Methods Enzymol. 346 466-500
Routier, F. H., Davies, M. J., Bergemann, K and Housell, E. F. 1997 ‘The glycosylation pattern of a humanized IgGl antibody (D1.3) expressed in CHO cells’ Glycoconjugate Journal 14 201-207
Salter, D. W. 1993 In: Manipulation of the Avian Genome’ (Etches, R. J. and Verrinder Gibbins, A. M. eds.) 135-150 CRC Press.
Salter, D. W. & Crittenden, L. B. 1989 ‘Artificial insertion of a dominant gene for resistance to avian leucosis virus into the germ line of the chicken’ Theor. Appl. Genet. 77 457-461.
Sang, H. and Perry, M. M. 1989 ‘Episomal replication of cloned DNA injected into the fertilised ovum of the hen, Gallus domesticus’ Molecular Reproductive Development 1(2) 98-106.
Sanjay, J. V., Stephens, E. B. and Narayan, O. 1996 ‘Lentiviruses’ 3^{rd} Edition. Vol.2. Lippincott-Raven Publishers, Philadelphia.
SCRIP, Jun. 8^{th} 2001 ‘Companies face up to biotech manufacturing issues’ p6 PJB Publications Ltd.
Speksnijder, G. and Ivarie, R. 2000 Poultry Science 79, 1430-1433.
Von Seggern, D. J., Huang, S., Fleck, S. K., Stevenson, S. C. and Nemerow, G. R. 2000 ‘Adenovirus vector pseudotyping in fiber-expressing cell lines: improved transduction of Epstein-Barr virus-transformed B cells’ J. Virol. 74(1) 354-36.
Weck, E. 1999 ‘Transgenic animals: Market Opportunities now a Reality’ D&MD Reports.
Yee, J. K., Friedmann,-T and Burns, J. C. 1994 “Generation of high-titer pseudotyped retroviral vectors with very broad host range” Methods of Cell Biology 43 Pt A 99-112.

Zhan, Y., Brady, J. H., Johriston, A. M. & Lew, A. M. 2000 ‘Predominant transgene expression in exocrine pancreas directed by the CMV promoter’ DNA Cell. Biol. 19 639-645

TABLE 1
PCR analysis of hatched chicks and germline transmission
from founder cockerels

| | | | Genome | | Germline | |
|---|---|---|---|---|---|---|
| Experiment: Construct | | | equivalents | | transmission | |
| (Viral titre) | | Bird No. | CAM | Semen | Transgenics/total | |
| 1. | pONY8.0cZ | 1-1 | 0 | 0.05 | 1/14 | (7%) |
| | 5 × 10⁷ T.U./ml | 1-2 | 0.01 | ♀ | — | |
| | | 1-3 | 0 | ♀ | — | |
| | | 1-4 | 0.01 | 0.5 | 16/55 | (29%) |
| | | 1-5 | 0.01 | 0.1 | nd | |
| 2. | pONY8.0cZ | 2-1 | 0.1 | ♀ | — | |
| | 5 × 10⁸ T.U./ml | 2-2 | 0.1 | 1.0 | 4/20 | (20%) |
| | | 2-3 | 0 | 0.01 | nd | |
| | | 2-4 | 0.1 | 0.5 | 19/67 | (28%) |
| | | 2-5 | 0 | ♀ | — | |
| | | 2-6 | 0.05 | ♀ | — | |
| | | 2-7 | 0.05 | ♀ | — | |
| | | 2-8 | 0.05 | 0.5 | 15/60 | (25%) |
| 3. | pONY8.4GCZ | 3-1 | 0 | 0.05 | 1/25 | (4%) |
| | 7.2 × 10⁸ T.U./ml | 3-2 | 0 | 0.05 | 3/64 | (5%) |
| | | 3-3 | 0.01 | ♀ | — | |
| | | 3-4 | 0.01 | 0.05 | 4/100 | (4%) |
| | | 3-5 | 0.01 | 0.1 | 9/82 | (11%) |
| | | 3-6 | 0.01 | ♀ | — | |
| 4. | pONY8.0G | 4-1 | 0.05 | 1.0 | 20/44 | (45%) |
| | 9.9 × 10⁹ T.U./ml | | | | | |

TABLE 2
Estimation of number of provirus insertions in the genome of G₁ birds

| | | Number of birds | | | | |
|---|---|---|---|---|---|---|
| | Total G₁ | with N insertions | | | | Total no. |
| Bird no. | analysed | 1 | 2 | 3 | 4 | independent insertions |
| 1-4 | 14 | 11 | 3 | 0 | 0 | 10 |
| 2-2 | 4 | 3 | 1 | 0 | 0 | 4 |
| 2-4 | 14 | 11 | 2 | 1 | 0 | 14 |
| 2-8 | 14 | 10 | 1 | 2 | 1 | 19 |

1. A method for the production of transgenic avians, the method comprising the step of using a lentivirus vector system to deliver exogenous genetic material to avian embryonic cells or cells of the testes.

2. A method as claimed in claim 1 wherein the lentivirus vector system includes a lentivirus transgene construct in a form which is capable of being delivered to and integrated with the genome of avian embryonic cells or cells of the testes.

3. A method as claimed in claim 2 wherein the lentivirus construct is injected into the subgerminal cavity of the contents of an opened egg which is then allowed to develop.

4. A method as claimed in claim 2 wherein the construct is injected directly into the sub-blastodermal cavity of an egg.

5. A method as claimed in claim 2 wherein the lentivirus construct transduces germ cells at high efficiency.

6. A method as claimed in claim 1 wherein the genetic material encodes a protein.

7. A transgenic avian produced by a method as claimed in claim 1.

8. A transgenic avian and subsequent transgenic offspring produced as the offspring of a transgenic avian as claimed in claim 7.

9. A method for the production of an heterologous protein in an avian, the method comprising the step of delivering genetic material encoding the protein within a lentivirus vector construct to avian embryonic cells so as to create a transgenic avian which expresses the genetic material in its tissues.

10. A method as claimed in claim 9 wherein the transgenic avian expresses the gene in its oviduct so that the translated protein becomes incorporated into eggs.

11. A method as claimed in claim 10 further comprising the step of isolating the protein from the eggs.

12. (canceled)

13. (canceled)

14. A method as claimed in claim 9 wherein the transgenic avian expresses the heterologous protein in specific tissues.

15. A method as claimed in claim 1 wherein the lentivirus is selected from the group consisting of EIAV, HIV, SIV, DIV and FIV.

16. A method as claimed in claim 2 wherein the construct includes suitable enhancer promoter elements for subsequent production of protein.

17. A method as claimed in claim 2 wherein particles of the vector construct are packaged to produce a vector with an envelope.

18. A method of determining the likelihood of expression of a protein in a transgenic avian, the method comprising the step of detecting expression of the protein in oviduct cells in vitro.

19. A method as claimed in claim 9 wherein expression of the genetic material is tissue-specific.

20. A method as claimed in claim 9 wherein the transgenic avian expresses the heterologous protein in egg white or yolk.

21. A method as claimed in claim 9 wherein the lentivirus is selected from the group consisting of EIAV, HIV, SIV, DIV and FIV.

22. A method as claimed in claim 9 wherein the construct includes suitable enhancer promoter elements for subsequent production of protein.

23. A method as claimed in claim 9 wherein particles of the vector construct are packaged to produce a vector with an envelope.
